(19) United States
(12) Patent Application Publication
(10) Pub. No.: US 2025/0339014 A1
(43) Pub. Date: Nov. 6, 2025

(54) HAND CONTROL VALVE FOR JOINT AND FRACTURE VISUALIZATION WITH A CANNULAE

(71) Applicant: Genesis Medical Devices LLC, Indialantic, FL (US)

(72) Inventors: Daniel Nick Segina, Satellite Beach, FL (US); James A. Proctor, JR., Indialantic, FL (US)

(21) Appl. No.: 19/188,102

(22) Filed: Apr. 24, 2025

Related U.S. Application Data
(60) Provisional application No. 63/641,751, filed on May 2, 2024.

(51) Int. Cl. A61B 1/00; A61B 1/015; A61B 1/04; A61B 1/317

(52) U.S. Cl. CPC A61B 1/00068; A61B 1/00128; A61B 1/015; A61B 1/042; A61B 1/317

(57) ABSTRACT

An arthroscopic apparatus includes an interface to a diagnostic sheath and a control valve mechanism. The control valve mechanism includes a control valve port, a control valve coupling, and an actuator. The actuator actuates passage of a substance between the port and the coupling, with the actuator further disposed relative to the sheath interface to allow for simultaneous manipulation of a position of the diagnostic sheath and actuation of the valve.

### CROSS REFERENCE TO RELATED APPLICATIONS

This application claims priority to co-pending U.S. Provisional Application Ser. No. 63/641,751 filed May 2, 2024 entitled “Hand Control Flow Valve for Joint and Fracture Visualization with a Cannula”, the entire contents of which are hereby incorporated by reference.

### TECHNICAL FIELD

The application relates to arthroscopic visualization of joint and/or bone structures.

### BACKGROUND

Current state-of-the-art for arthroscopic visualization of joint and/or bone structures revolves around the use of a camera system in conjunction with a separate fluid pump and fluid evacuation mechanism. Technological advancements have provided for the ability to downsize the instrumentation and have the capacity to simplify visualization the route the human body.

An example of this technology is a nano needle arthroscopic device available from Arthrex, Inc. of Naples, Florida. This 1.9 mm scope is coupled to a pump that allows for pressure control in the traditional “wet” arthroscopy. This wet procedure revolves around fluid pressurization of a contained space followed by the insertion of a arthroscopic camera for visualization of structures and potential resection/repair of said structures.

The integrity of the contained space is critical for visualization and the maintenance of a fluid environment. Additionally, modulation of fluid pressure is also critical to ensure optimal visualization as well as prevent potential patient harm. Noting the crucial nature of fluid pressure management, sophisticated pump and pressure monitoring devices are necessary particularly in the operative theater. This technology has been validated for decades as the standard of care for joint visualization.

Arthroscopic visualization in fracture care has also been well-established. This technology does allow for the extension of visualization, particularly with articular reconstruction.

At least common challenges are commonly recognized in the application of arthroscopic visualization to fracture care scenarios. First, structural integrity of the patient has been disrupted with the fracturing of the bone, the articular surface, as well as disruption of the joint capsule. Additionally, surgical repair typically involves the opening of these structures, additionally eliminating the integrity to contain fluid that would be necessary for a typical “wet” arthroscopic procedure.

An additional application of arthroscopic visualization is commonly described as “dry” arthroscopy since it does not require a continually contained space. This technique is applicable to clinical situations where the integrity of the structure, particularly the joint space, has been violated, and the capacity to contain fluid does not exist. In these situations, the “wet” scope approach is significantly limited as a direct result of the loss of integrity of the structures. Although the “dry” arthroscopic technique does eliminate the need for pressurized fluid flow, it is limited by the lack of ability to maintain a constant and clean visual field. Additionally, material and localized fluid, particularly blood, frequently obscures the visual field of the arthroscope and limits its clinical utility.

One ad-hoc prior art solution using a combination of standard components to overcome the challenges of both “wet”, and “dry” arthroscopic visualization can be referred to as a modulated, wet/dry scope, or a hybrid scope procedure.

An example of a known design is depicted in FIG. 1. This includes a combination of a diagnostic sheath 101 with a stopcock valve 107. Additional details for the diagnostic sheath 101 include: a camera interface 102, an elongated camera sheath 103, and a camera sheath exit portal 104. Camera interface 102 accepts the camera and a light source, allowing for protected insertion into various anatomic structure and spaces. Additionally, camera sheath 103 contains potential space for the inflow and egress of fluid. Additional details may include a sheath flow interface 105, providing access for either inflow or outflow through an elongated camera sheath 103. Stop cock connector 106 provides a coupling mechanism to the diagnostic sheath 101. Stop cock valve housing 107 serves to contain stopcock valve mechanism(s). Stopcock valve control 108 and stop cock inflow portal 109 are also provided.

### BRIEF SUMMARY

Limitations of the ad-hoc approach shown in FIG. 1 include but are not limited to (a) the fixed position of the stopcock valve, which lacks any capacity to accommodate graduated flow and (b) the inability to accommodate a default position when mechanical control is removed.

Additionally, the valve requires external manipulation, which detracts from the camera operator's capacity to control the camera during surgical, visualization and simultaneously control, graduated, inflow, and or egress of fluid. This is usually accommodated by additional operating room personnel managing the stopcock control valve thus complicating the visualization portion of the procedure.

One objective herein is to provide an apparatus for single operator control of inflow and/or evacuation of fluid either combined or individually. This challenge is overcome with the use of intermittent injection and evacuation of fluid to clear the tip of the camera restoring optimal visualization. Additionally, the capacity for graduated manual control of the inflow, and or egress of fluid is significantly enhanced with a manual control valve/mechanism that accommodates a default setting once valve control input is removed.

More particularly the techniques described herein relate to an arthroscopic apparatus including: a sheath interface to a diagnostic sheath; and a control valve mechanism including a control valve port; a control valve coupling; and an actuator, the actuator configured to actuate passage of a substance between the port and the coupling, the actuator further disposed relative to the sheath interface to allow for simultaneous manipulation of a position of the diagnostic sheath and actuation of the valve.

The diagnostic sheath may further include a flow interface configured to provide access to a substance flow; and a camera interface.

The actuator and the diagnostic sheath may be provided as an integrated unit.

The control valve mechanism may be mechanically coupled to the diagnostic sheath via a sheath interface.

The sheath interface may include a coupler adapted to engage a fluid port on the diagnostic sheath.

The apparatus may be configured to balance a mechanical force applied to the actuator along at least one axis, to prevent disruption of an orientation of the diagnostic sheath.

The control valve mechanism is configured for bi-directional flow of the substance between the port and the coupling.

The substance is a fluid, a gas, or other flowable material.

The actuator may include a motorized valve that operates by manual or voice activated input, or through a solenoid, a pressure sensor mechanism, a relay, or some other electromagnetic or mechanical means.

A second control valve mechanism may also be provided. If so configured, this includes a second control valve port; a second valve coupling; and a second actuator configured to operate the second control valve mechanism independently.

A first one of the valve mechanisms may be coupled to a suction source, and a second one of the valve mechanisms is coupled to an irrigation source.

A first one of the valve control ports may be coupled to a suction source, and second of the valve control ports coupled to an irrigation source.

The control valve mechanism may provide directional flow.

The control valve mechanism may be configured to provide a default setting.

The default setting may be initiated by stimulus input at the actuator; b. continuous suction; c. continuous irrigation; or d. initiated by removal of manual input to the actuator.

The actuator may be configured for manual manipulation that provides graduated flow control.

The control valve mechanism may be bidirectional; in which case a second control valve mechanism is provided with a second control port; and wherein both control valves are controlled by the actuator.

Furthermore, pressing the actuator in one direction may gradually engage only a first one of the valves, while remaining unengaged with a second one of the valves; and pressing the actuator in an opposite direction, may gradually engage the second one of the valve, while not engaging the first valve.

### BRIEF DESCRIPTION OF THE DRAWINGS

FIG. 1 depicts a prior art set up for arthroscopic visualization.

FIG. 2A-1 shows an example of an improved diagnostic sheath having an enhanced flow control valve.

FIG. 2A-2 is an alternative embodiment of the enhanced flow control valve.

FIG. 2A-3 shows another example of an enhanced control valve.

FIGS. 2B, 2C, 2D, 2E, 2F and 2G depict further alternative embodiments of the flow control valve.

FIGS. 3A and 3B depict variations in position of a poppet design for a valve mechanism.

FIGS. 4A, 4B and 4C depict alternative embodiments for the flow control valve.

FIGS. 5A, 5B, 5C and 5D depict an alternative design for the control valve.

FIGS. 6A and 6B show an implementation of a poppet valve.

FIG. 7A depicts an example integrated diagnostic sheath.

FIG. 7B depicts another example of an integrated diagnostic sheet.

### DETAILED DESCRIPTION

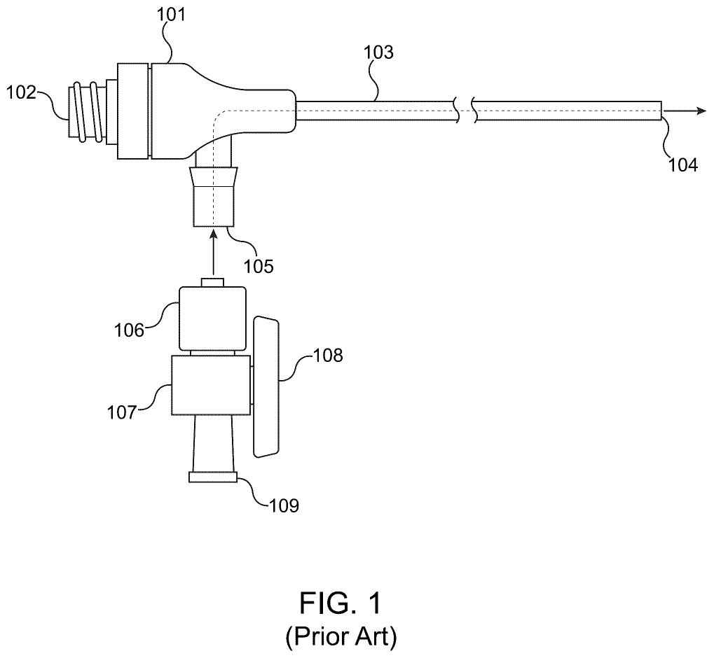

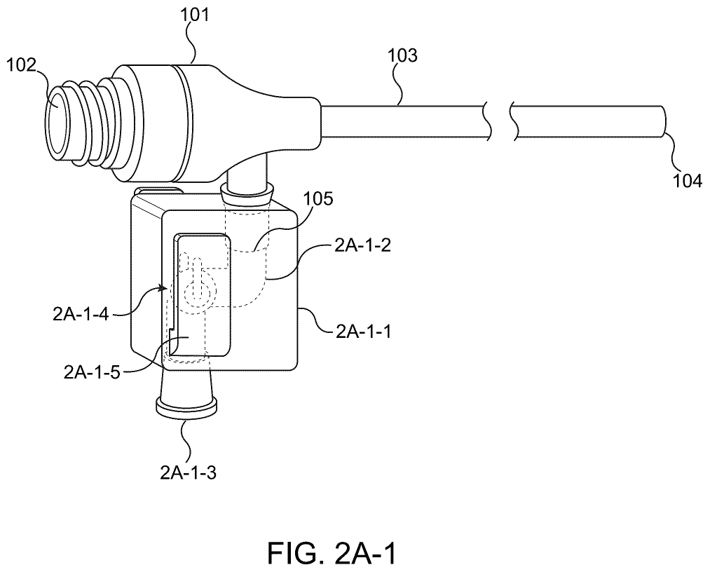

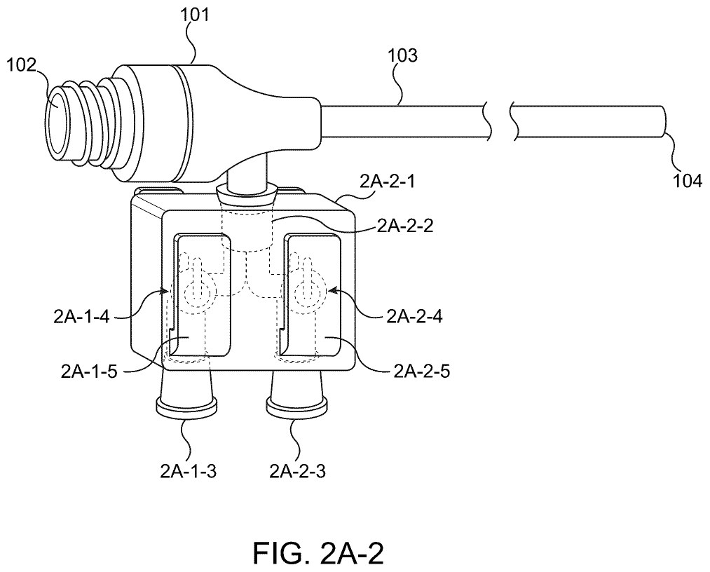

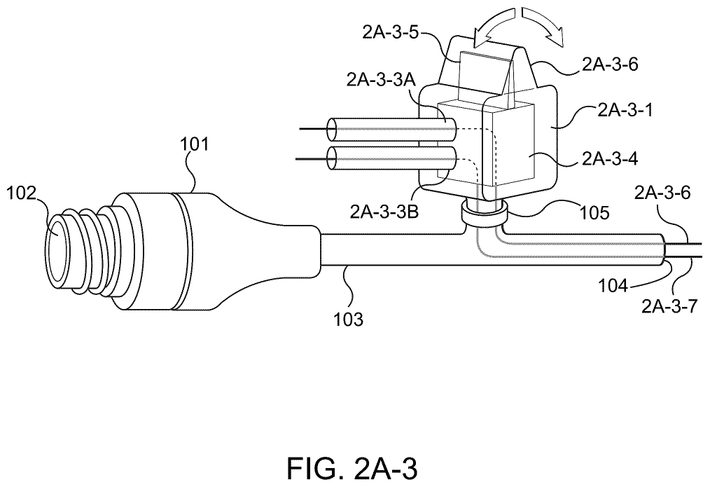

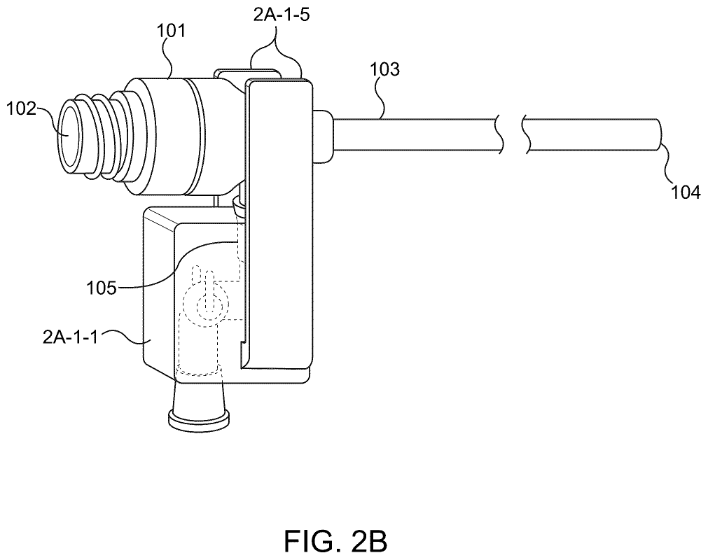

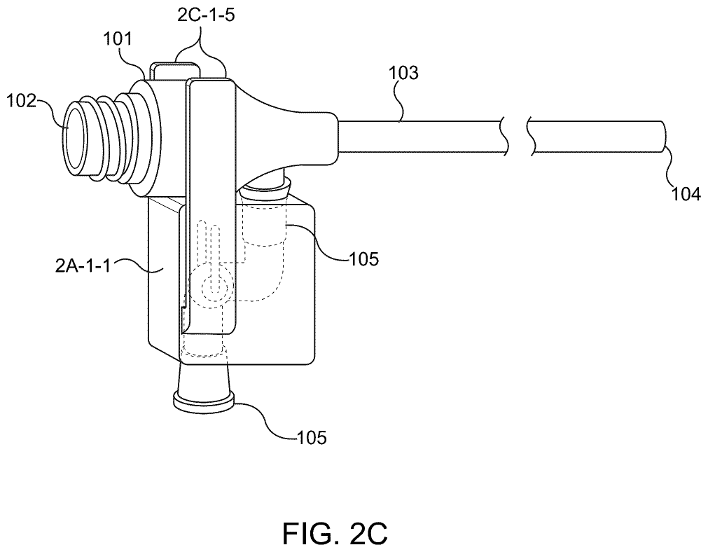

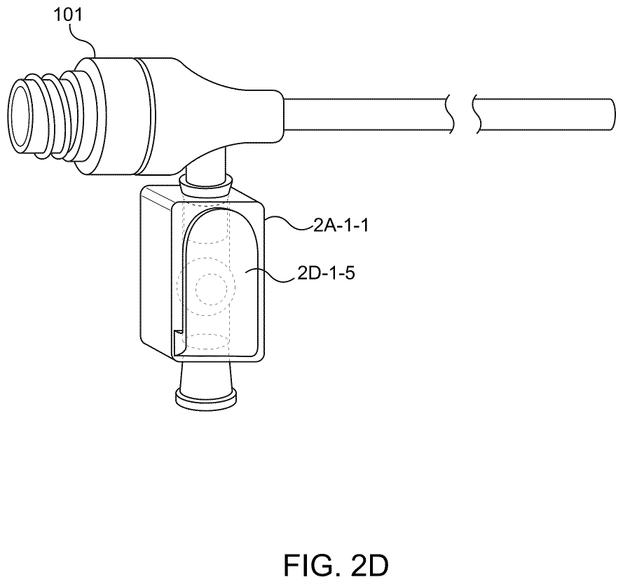

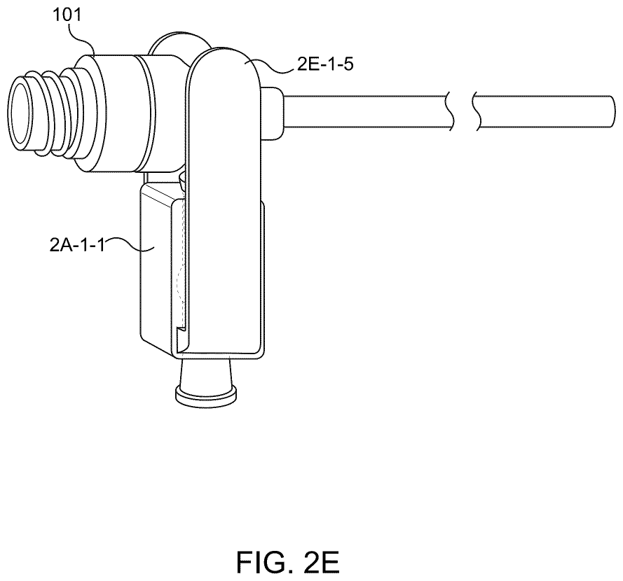

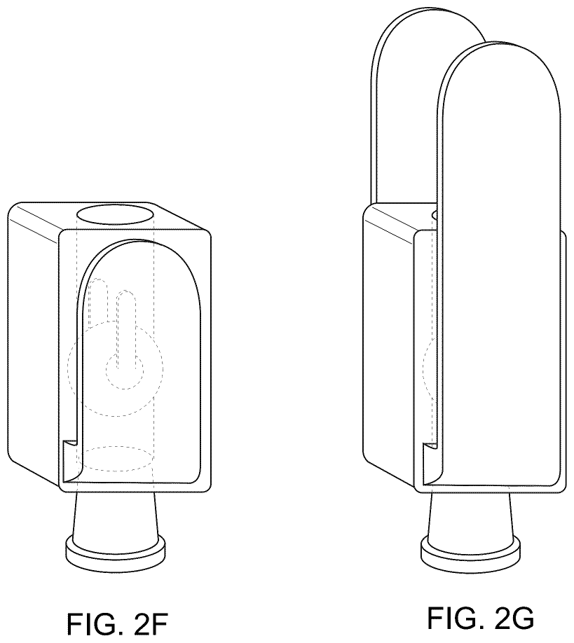

Depicted in FIG. 2A-1 is an example diagnostic sheath 101 coupled with one embodiment of an enhanced control valve 2A-1-1 according to the teachings herein. The enhanced control valve 2A-1-1 interfaces with the diagnostic sheath 101 at a sheath flow interface 105. A bi-directional valve control port 2A-1-2 interfaces with the diagnostic sheath 101 through a sheath flow interface 105. Reference numeral 2A-1-3 depicts a bi-directional valve coupling portal, reference numeral 2A-1-4 depicts the enhanced control valve mechanism, and reference numeral 2A 1-5 depicts a valve actuator. One embodiment of this actuator 2A-1-5 is a direct manual control. Additional embodiments may include a motorized valve mechanism. Control of this motorized valve mechanism may either be manual or voice activated, or actuator controlled. This may be achieved through a solenoid, a pressure sensor mechanism, a relay, or through other electromagnetic and/or mechanical means (not shown in the figures).

FIG. 2A-2 is an additional embodiment of an enhanced flow control valve indicated as item 2A-2-1. Here, item 2A-2-2 is a bi-directional valve control port interfacing with two separate enhanced control valve mechanisms, 2A-1-4 and 2A-2-4, allowing for directed flow through either valve control mechanism. Item 2A-2-5 depicts a valve actuator similar to valve actuator 2A-1-5. A second bi-directional valve coupling portal 2A-2-3 is similar to portal 2A-1-3. The addition of a second enhanced control valve provides for the capacity to simultaneously regulate the passage, including both inflow and egress, of a substance, either via gravity or active suction. Although the following description explains that one or more valves control the flow of a fluid, it should be understood that in some embodiments, the valve(s) may control the flow other substances such as a gas, or still other substances even solids such as a flowable particulate.

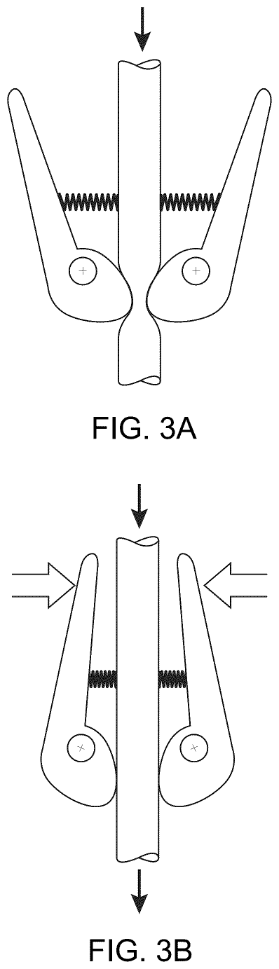

FIG. 2A-3 depicts an additional embodiment of an enhanced control valve 2A-3-1, this implementation comprising a directional integrated valve 2A-3-4, and having two external ports 2A-3-3A and 2A-3-3B. In some embodiments, port 2A-3-3A may be coupled to an irrigation source and port 2A-3-3B may be coupled to suction source. The passage (or flow) through these respective ports are depicted as 2A-3-6 and 2A-3-7. In one embodiment, integrated valve 2A-3-4 may be comprised of valves 2A-1-4 and 2A-2-4 along with a controlled port 2A-2-2, and controlled by a single actuator 2A-3-6. In some embodiments, a protective covering 2A-3-5 encompasses actuator 2A-3-6. In other embodiments, a single valve mechanism may allow for bi-directional control between the two interface ports 2A-3-3A and 2A-3-3B, allowing for bidirectional control to sheath flow interface A105.

Another embodiment may include various default valve control settings, such as when a stimulus input to actuator 2A-3-6 is provided by the operator as a default setting. Other default settings may include continuous suction from one input or continuous irrigation from another input port, while in other embodiments, a combination of the two may be provided.

Mechanical manipulation by the operator of actuator 2A-3-6 through protective covering 2A-3-5 can allow for directed graduated flow through bi-directional valve 2A-3-4. One embodiment of bi directional valve 2A-3-4 includes two separate individual valve mechanisms controlled through a common actuator 2A-3-6. The movement of the actuator 2A-3-4, through protective covering 2A-3-5 in a forward direction, may allow for varying flow rates of fluid, while movement of the actuator in the reverse direction may allow for varying intensity of suction strength.

Movement of the actuator(s) may be accomplished through various mechanisms known to one of skill in the art. For example, pressing the actuator in one direction, may gradually engage only one of the valves, while remaining unengaged with the other valve, whereas pressing the actuator in the opposite direction, may engage the opposite valve gradually while not engaging the prior valve, thus allowing the described behavior.

In some embodiments, removal of manual input will allow for the return of the bi-directional valve 2A-3-4, to a default setting.

An additional embodiment of valve 2A-3-4 may include a single valve housing, utilizing a gate having offset holes, allowing for one the ports 2A-3-3 A or 2A-3-3B to be engaged separately from the other port. Other internal valve mechanisms, known for use in these application, may be employed here, as well as a closure, element or obturator. These may also include variations of gate, ball, plug, disk, and poppet elements.

FIGS. 2B through 2G depict alternative embodiments of the flow control valve in which flow control valve 2A-1-1 includes variations in actuator arm design, as well as actuator arm placement, as well as alternative valve configurations.

FIG. 2B, the placement of the actuator arms 2A-1-5 are symmetrically on other side of sheath 103, and extended so as to allow for a user to both hold the sheath and manipulate and operate the actuators. The extension of the actuator arms for example additionally allows for operation of the device with either left or right hand operation in one embodiment.

Referring now of FIG. 2C, the actuators arms are placed in an alternative position, more distal to its previous placement and inline with the value mechanism.

FIG. 2D depicts an embodiment including a simplified plunge valve implementation in which the actuator 2E-01-5 interfaces directory with the value, in one embodiment.

FIG. 2E depicts the mechanism of FIG. 2D, with extended actuator 2E-1-5 for improved ergonomics and joint manipulation of the sheath and apparatus with reduced impact to the sheath alignment when operating the actuator due to relative applied force. With the depicted extended actuator 2E-1-5, forces applied to the actuator are now balanced, preventing or at least reducing disruption of the orientation and/or alignment of the diagnostic sheath 101.

FIG. 2F and FIG. 2G are respective alternative depictions of FIGS. 2D and 2E without the diagnostic sheath apparatus interfaced to the flow control value assembly, with FIG. 2G again showing the version with an extended actuator.

FIGS. 3A and 3B depict variations in position of a poppet design for use in with the various valve mechanisms allowing for a default position with the valve off. In these figures, applying pressure to the valve input allows for graduated flow through the valve. As depicted, the spring-biased lever(s) (the actuators) control the amount of pressure applied to the valve control allowing for variation in flow (in or out), and assuming a default position when pressure to the actuator is removed in a poppet style value. As one example associated with FIG. 3A, when no external pressure is applied to the actuator arms, the value assumes a default off position with the ends of each respective actuator arm applied a closing pressure to flexible coupling hose pinched between them in a poppet style value mechanism. Referring to FIG. 3B, when pressure is applied to the distal ends of the actuator arm (depicted as arrows in the figure), the pinching mechanism interfacing with the flexible hose begins to open gradually allowing progressively more flow though the hose as additional pressure is applied to open the valve.

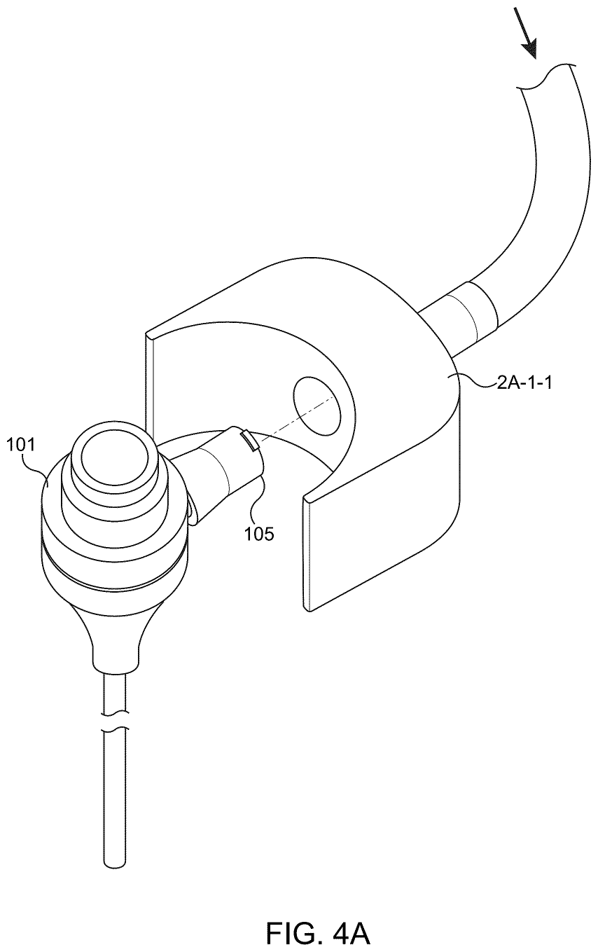

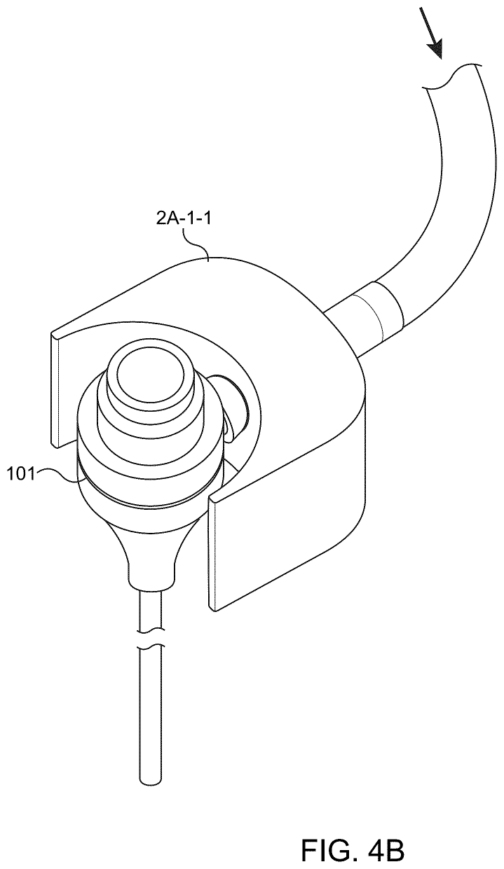

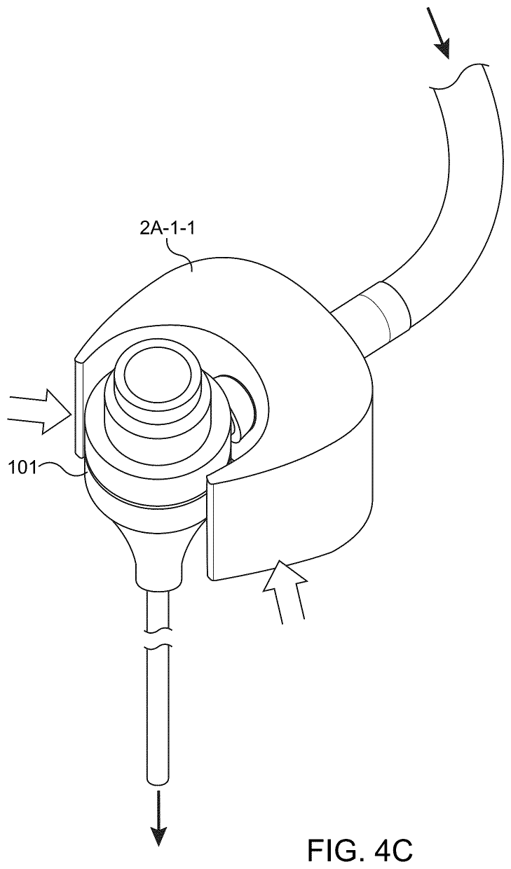

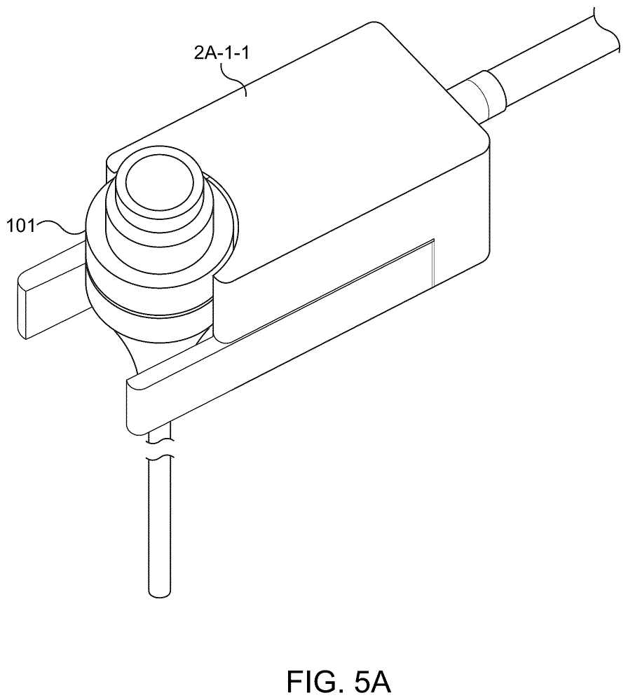

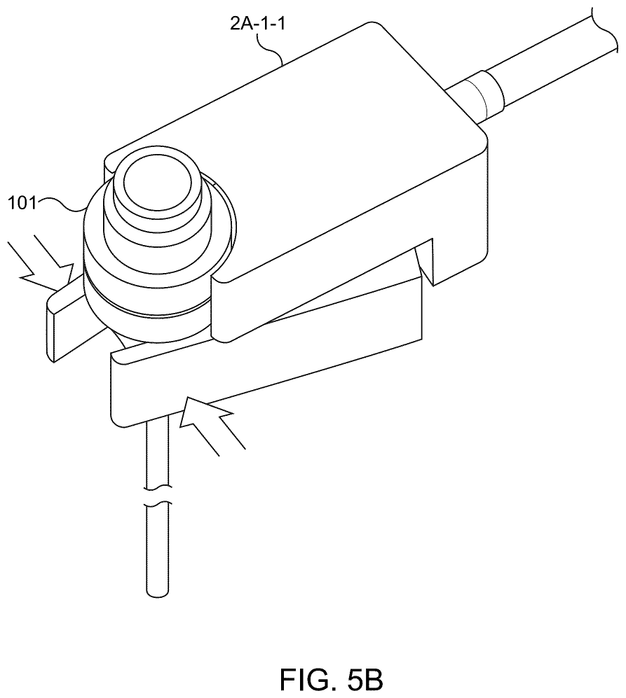

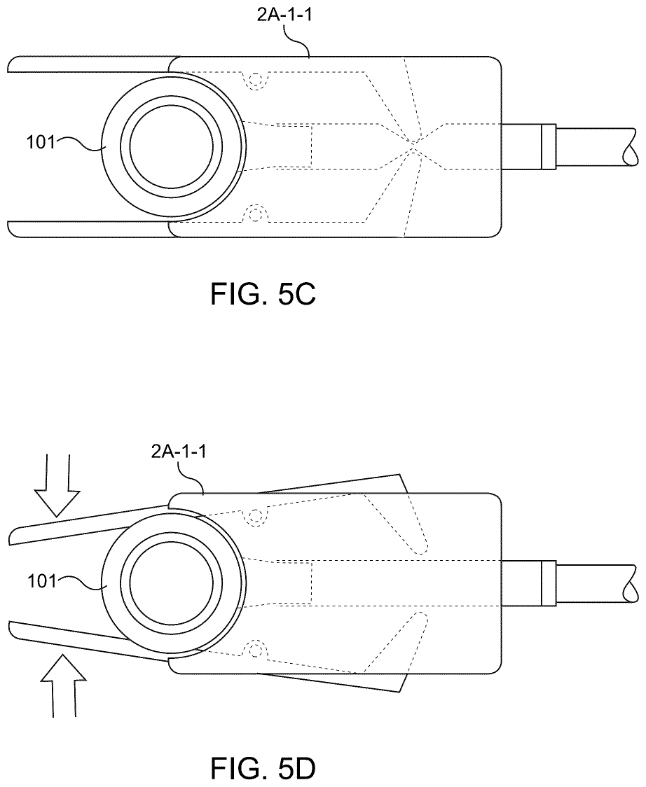

FIGS. 4A, 4B and 4C depict alternative embodiments of the flow control valve 2A-1-1 utilizing a single input port, and provide for a ergonomically varying interface (e.g., with extended actuator that at least partially encircles the diagnostic sheath 101). Other embodiments may include multiple ports as depicted in FIG. 2A-2. FIG. 4B depicts a particular situation with no manual input to flow control valve 2A-1-1 resulting in no flow or suction to the input ports coupled to diagnostic sheath 101. FIG. 4C depicts operator input to flow control valve 2A-1-1, allowing for controlled inflow and/or outflow via the diagnostic sheath 101. The implementation of the mechanism of FIGS. 4A, 4B and 4C is depicted in FIGS. 5A, 5B, 5C, and 5D in some embodiments.

FIGS. 5A, 5B, 5C and 5D depict still other alternative embodiments of control valve 2A-1-1. FIGS. 5A and 5C show the valve in the off position as a default. FIGS. 5B and 5D demonstrate the opening of the valve 2A-1-1 with applied manual pressure. An alternative embodiment may apply to input ports having to separate actuator arms, so that the operator may open one or the other valve. In further embodiments, a single set of actuator arms may operate both valves, mutually exclusively, allowing for graduated, controlling of one or the other port. As one example associated with FIG. 5C, when no external pressure is applied to the actuator arms, the valve(s) assume a default off position with the ends of each respective actuator arm applied a cloison pressure to flexible coupling hose pinched between them in a poppet style value mechanism. This similar to the operation depicted in FIG. 3A. As depicted, the spring-biased lever(s) (the actuators) control the amount of pressure applied to the valve control allowing for variation in flow (in or out), and assuming a default position when pressure to the actuator is removed. As one example associated with FIGS. 5A and 5C, when no external pressure is applied to the actuator arms, the valve assumes a default off position with the ends of each respective actuator arm applied a closing pressure to flexible coupling hose pinched between them in a poppet style value mechanism. Referring to FIGS. 5B and 5D, when pressure is applied to the distal ends of the actuator arm (depicted as arrows in the figure), the pinching mechanism interfacing with the flexible hose begins to open gradually allowing progressively more flow through the hose as additional pressure is applied to open the valve.

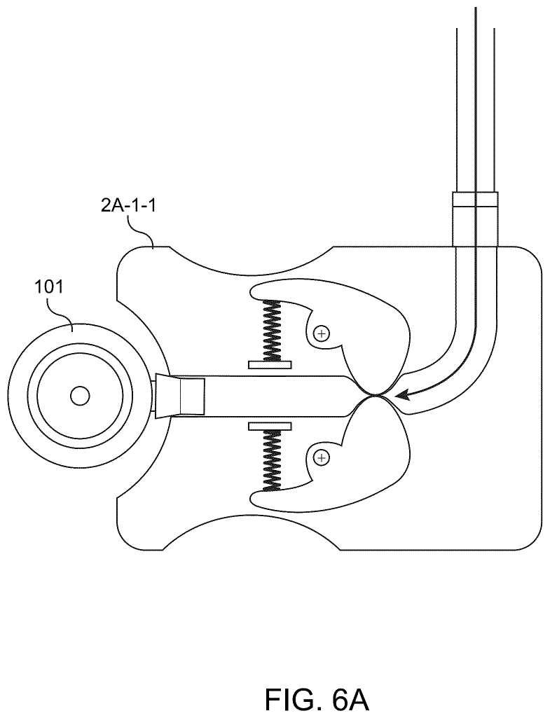

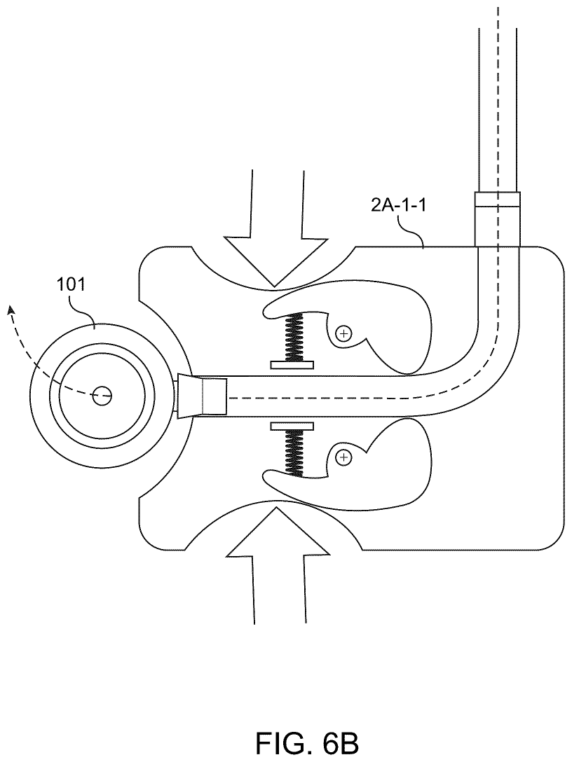

FIGS. 6A and 6B show another poppet valve design, associated with the flow control valve of FIG. 4A-C. FIG. 6A depicts a flexible housing (such as silicon, rubber, plastic, foam, etc.) 2A-1-1 with internal poppet style value mechanisms as described associated with FIGS. 3A and 3B, as a alternative example embodiment to that depicted in FIGS. 4A, 4B and 4C. In an alternative embodiment the structures of FIG. 5A through 5D can be including internally to the housing 2A-1-1 of FIG. 6A. FIG. 6B depicts an embodiment of FIG. 6A, with manual pressure applied to the actuators as shown in the arrows and applied force operating the value mechanism through the flexible covering 2A-1-1, allowing for flow to the diagnostic sheath 101.

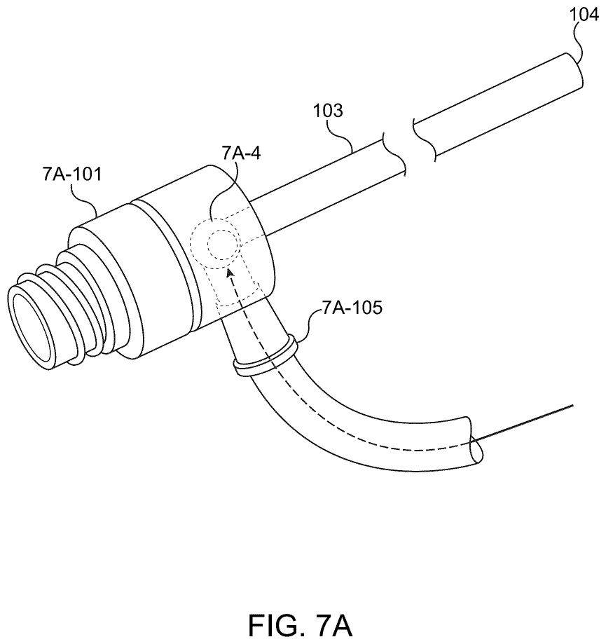

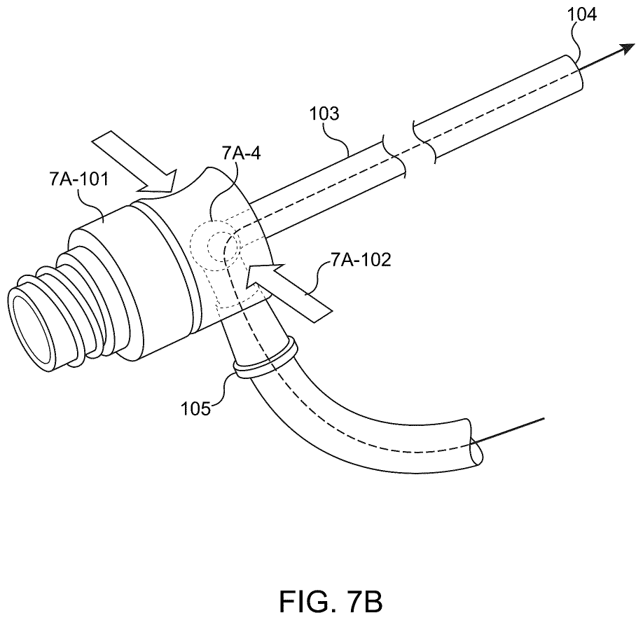

FIG. 7A depicts an integrated diagnostic sheath 7A-101 and enhanced flow control valve 7A-4, allowing for inflow and or outflow coupling through enhance sheath interface 7A-105 in the default off state.

FIG. 7B depicts an integrated diagnostic sheath 7A-101 and enhance flow control valve 7A-4 along with directed manual input point 7B-102, allowing for controlled flow through enhanced flow control valve 7A-4. Additional embodiments of the integrated sheath 7A-101 may allow for multiple ports and actuator controls as depicted in FIG. 2A-2-2. Multiple directed manual input access points 7B 102 may be placed axially or radially to allow for varying control of individual actuators.

### Further Implementation Options

The foregoing description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to the communication systems and networks, have not been shown or described in order to avoid unnecessarily obscuring descriptions of the embodiments. Additionally, the various embodiments may be methods, systems, media, or devices. Accordingly, the various embodiments may be entirely hardware embodiments, entirely software embodiments, or embodiments combining software and hardware aspects.

Throughout the specification, claims, and drawings, the following terms take the meaning explicitly associated herein, unless the context clearly dictates otherwise. The term “herein” refers to the specification, claims, and drawings associated with the current application. The phrases “in one embodiment,” “in another embodiment,” “in various embodiments,” “in some embodiments,” “in other embodiments,” and other variations thereof refer to one or more features, structures, functions, limitations, or characteristics of the present disclosure, and are not limited to the same or different embodiments unless the context clearly dictates otherwise. As used herein, the term “or” is an inclusive “or” operator, and is equivalent to the phrases “A or B, or both” or “A or B or C, or any combination thereof,” and lists with additional elements are similarly treated. The term “based on” is not exclusive and allows for being based on additional features, functions, aspects, or limitations not described, unless the context clearly dictates otherwise. In addition, throughout the specification, the meaning of “a,” “an,” and “the” include singular and plural references.

The above description has particularly shown and described example embodiments. However, it will be understood by those skilled in the art that various changes in form and details may be made therein without departing from the legal scope of this patent as encompassed by the appended claims.

1. An arthroscopic apparatus comprising:
a sheath interface to a diagnostic sheath; and
a control valve mechanism comprising:
a control valve port;
a control valve coupling; and
an actuator, the actuator configured to actuate passage of a substance between the port and the coupling, the actuator further disposed relative to the sheath interface to allow for simultaneous manipulation of a position of the diagnostic sheath and actuation of the valve.

2. The apparatus of claim 1 wherein the apparatus additionally comprises the diagnostic sheath, and wherein the diagnostic sheath further comprises:
a flow interface configured to provide access to a substance flow; and
a camera interface.

3. The apparatus of claim 2 wherein the actuator and the diagnostic sheath are an integrated unit.

4. The apparatus of claim 1 wherein the control valve mechanism is mechanically coupled to the diagnostic sheath via a sheath interface.

5. The apparatus of claim 4 wherein the sheath interface is a coupling adapted to engage a fluid port on the diagnostic sheath.

6. The apparatus of claim 1 wherein the actuator is further configured to balance mechanical force applied to the actuator to avoid disruption of an orientation and/or alignment of the diagnostic sheath.

7. The apparatus of claim 1 wherein the control valve mechanism is configured for bi-directional flow of the substance between the port and the coupling.

8. The apparatus of claim 1 wherein the substance is a fluid, a gas, or other flowable material.

9. The apparatus of claim 1 wherein actuator comprises a motorized valve that operates by manual or voice activated input, or through a solenoid, a pressure sensor mechanism, a relay, or some other electromagnetic or mechanical means.

10. The apparatus of claim 1 additionally comprising:
a second control valve mechanism comprising:
a second control valve port;
a second valve coupling; and
a second actuator configured to independently operate the second control valve mechanism.

11. The apparatus of claim 10 wherein a first one of the valve mechanisms is coupled to a suction source, and a second one of the valve mechanisms is coupled to an irrigation source.

12. The apparatus of claim 1 additionally comprising:
a second control valve port; and
a first of the valve control ports is coupled to a suction source, and second of the valve control ports is coupled to an irrigation source.

13. The apparatus of claim 12 wherein the control valve mechanism is configured for directional flow of fluid.

14. The apparatus of claim 1 wherein the control valve mechanism is configured to provide a default setting.

15. The apparatus of claim 14 wherein the default setting is:
a. initiated by stimulus input at the actuator;
b. continuous suction;
c. continuous irrigation; or
d. initiated by removal of manual input to the actuator.

16. The apparatus of claim 1 wherein the actuator is configured for manual manipulation that provides graduated flow control.

17. The apparatus of claim 1 wherein the control valve mechanism is bidirectional and the apparatus additionally comprises:
a second control valve mechanism having a second control port; and
wherein both control valves are controlled by the actuator.

18. The apparatus of claim 10 wherein:
pressing the actuator in one direction gradually engages only a first one of the valves, while remaining unengaged with a second one of the valves; and
pressing the actuator in an opposite direction, gradually engages the second one of the valve, while not engaging the first valve.
